(19) United States
(12) Patent Application Publication
(10) Pub. No.: US 2008/0139660 A1
(43) Pub. Date: Jun. 12, 2008

(54) Tablets comprising entacapone and crospovidone

(76) Inventor: Bernard Charles Sherman, Toronto (CA)

Correspondence Address: NIXON & VANDERHYE, PC, 901 NORTH GLEBE ROAD, 11TH FLOOR, ARLINGTON, VA, 22203, US

(21) Appl. No.: 11/987,721

(22) Filed: Dec. 4, 2007

(30) Foreign Application Priority Data
Dec. 8, 2006 (CA) 2,572,004

(51) Int. Cl. A61K 31/165

(52) U.S. Cl. 514/619

(57) ABSTRACT

Pharmaceutical tablets comprising entacapone and crospovidone.

### BACKGROUND OF THE INVENTION

The present invention relates to a pharmaceutical tablet comprising entacapone, and crospovidone as a dissolution enhancing agent.

Entacapone is described in U.S. Pat. No. 5,446,194 as a catechol-O-methyltransferase (COMT) inhibitor.

It is desirable that entacapone be released from a tablet as soon as possible after it is ingested. This can normally be achieved by including in the tablet a dissolution enhancing agent. There is a vast selection of different dissolution enhancing agents on the market, including disintegrants, which have different chemical and physical characteristics. However, it has been found that it is difficult to formulate tablets comprising entacapone that exhibit rapid disintegration and dissolution.

U.S. Pat. No. 6,599,530 discloses that croscarmellose sodium is a superior disintegrant to be used in a tablet comprising entacapone. Entacapone tablets made according to the teaching of U.S. Pat. No. 6,599,530 are sold in the United States under the tradename Comtan™.

However, croscarmellose sodium detracts from the hardness of a tablet, thus requiring a more complex formulation than would be possible using a disintegrant that does not detract from hardness.

### DESCRIPTION OF THE INVENTION

It has been found that, for tablets comprising entacapone, use of crospovidone as a disintegrant enables disintegration and dissolution at least as rapid as that of Comtan™ tablets. Moreover, because inclusion of crospovidone does not detract from tablet hardness, use of crospovidone enables simplification of the formulation. In fact, it is possible to make hard tablets comprising nothing other than entacapone, crospovidone, and a lubricant. Tablets of the present invention thus comprise entacapone and crospovidone.

In tablets of the present invention, the amount of crospovidone will be from 6% to 85% of the tablet by weight, preferably from 20% to 75% by weight, and most preferably from 40% to 70% by weight.

The tablets will also contain a lubricant, such as, for example, stearic acid.

The invention will, be further clarified by the following example, which is intended to be exemplary and not limiting.

### EXAMPLE 1

Entacapone, crospovidone and stearic acid were mixed in proportions as follows:

| | |
|---|---|
| Entacapone | 40.0% |
| Crospovidone | 57.0% |
| Stearic Acid | 3.0% |
| | 100.0% |

The powder mixture was compacted, the compacted material was milled into granules, and the granules were recompressed into tablets of weight 500 mg, such that each tablet comprised 200 mg of entacapone.

Tablets of this example were compared to Comtan™ tablets for dissolution rate in USP apparatus 2, at 50 rpm, in 900 mL of phosphate buffer, pH5.5. It was found that, for tablets of this example, over 80% was dissolved in less than 20 minutes, and that the dissolution profile was very similar to that of Comtan™ tablets.

1. A tablet comprising entacapone, crospovidone and a lubricant, wherein the amount of crospovidone is from 6% to 85% of the tablet by weight.

2. A tablet of claim 1, wherein the amount of crospovidone is from 20% to 75% of the tablet by weight.

3. A tablet of claim 2, wherein the amount of crospovidine is from 40% to 70% of the tablet by weight.

4. A tablet of claim 1, wherein the lubricant is stearic acid.
